(19) United States
(12) Patent Application Publication
(10) Pub. No.: US 2015/0011889 A1
(43) Pub. Date: Jan. 8, 2015

(54) ULTRASONIC PROBE AND MANUFACTURING METHOD THEREOF

(71) Applicant: Samsung Medison Co., Ltd., Gangwon-Do (KR)

(72) Inventor: Sung Jae LEE, Seoul (KR)

(21) Appl. No.: 14/326,167

(22) Filed: Jul. 8, 2014

(30) Foreign Application Priority Data
Jul. 8, 2013 (KR) 10-2013-0079759

(51) Int. Cl. A61B 8/00

(52) U.S. Cl. CPC A61B 8/4444; A61B 8/546
USPC 600/459; 29/595

(57) ABSTRACT

An ultrasonic probe manufactured using graphene or graphite, and a manufacturing method thereof, the ultrasonic probe including a matching layer, a transducer layer provided at a rear surface of the matching layer, and a backing layer provided at a rear surface of the transducer layer, wherein the ultrasonic probe further includes at least one sheet that is formed of graphene and provided on at least one of a front surface of the matching layer, in between the matching layer and the transducer layer, in between the transducer layer and the backing layer, a rear surface of the backing layer, and lateral sides of the matching layer, the transducer layer and the backing layer.

### CROSS-REFERENCE TO RELATED APPLICATION

This application claims the benefit of Korean Patent Applications No. 2013-0079759, filed on Jul. 8, 2013 in the Korean Intellectual Property Office, the disclosure of which is incorporated herein by reference.

### BACKGROUND

1. Field

Embodiments of the present disclosure relate to an ultrasonic probe for generating an internal image of an object by use of ultrasonic waves.

2. Description of the Related Art

An ultrasonic diagnosis apparatus is an apparatus that acquires an image regarding soft tissue or a blood stream by irradiating ultrasonic waves toward a target region of the interior of a body of an object from the surface of the object, and by non-invasively receiving a reflected ultrasonic signal (ultrasonic echo signal).

The ultrasonic diagnosis apparatus is small and inexpensive when compared to other image diagnosis apparatuses, such as an X-ray diagnosis apparatus, a computerized tomography (CT) scanner, a magnetic resonance imager (MRI), and a nuclear medicine diagnosis apparatus, and is capable of displaying a diagnosis image in real time. In addition, the ultrasonic diagnosis apparatus has a high safety against radiation exposure, and is thus widely used for heart diagnosis, celiac diagnosis, urinary diagnosis, and obstetrics and gynecology.

The ultrasonic diagnosis apparatus includes an ultrasonic probe transmitting ultrasonic waves to an object and receiving an ultrasonic echo signal reflected by the object so as to acquire an image of the interior of the object.

The ultrasonic probe includes a transducer layer in which a piezoelectric material vibrates to execute conversion between an electrical signal and an acoustic signal, a matching layer reducing an acoustic impedance difference between the traducer layer and the object so as to maximally transmit ultrasonic waves generated from the transducer layer to the object, a lens concentrating ultrasonic waves proceeding in the forward direction of the transducer layer on a predetermined point, and a backing layer preventing ultrasonic waves from proceeding in the backward direction of the transducer layer to prevent image distortion.

### SUMMARY

Therefore, it is an aspect of the present disclosure to provide an ultrasonic probe that is manufactured using graphene or graphite, and a manufacturing method thereof.

Additional aspects of the disclosure will be set forth in part in the description which follows and, in part, will be obvious from the description, or may be learned by practice of the disclosure.

In accordance with an aspect of the present disclosure, an ultrasonic probe includes a matching layer, a transducer layer, and a backing layer. The transducer layer may be provided at a rear surface of the matching layer. The backing layer may be provided at a rear surface of the transducer layer. The ultrasonic probe may further include at least one sheet that is formed of graphene and provided on at least one of a front surface of the matching layer, in between the matching layer and the transducer layer, in between the transducer layer and the backing layer, a rear surface of the backing layer, and lateral sides of the matching layer, the transducer layer and the backing layer.

The ultrasonic probe may further include a signal line connected to the at least one sheet. The signal line may transmit heat sensed from the sheet toward a backend of the ultrasonic probe so as to check a degree of heat emission of the ultrasonic probe.

At least one of the backing layer and the matching layer may be formed of graphene.

The ultrasonic probe may further include a heat sink provided at the rear surface of the backing layer so as to dissipate heat generated from the ultrasonic probe to the outside.

The at least one sheet may extend to the heat sink so as to make thermal contact with the heat sink, and transfers absorbed heat to the heat sink.

The ultrasonic probe may further include at least one heat radiation plate that thermally connects the at least one sheet to the heat sink such that heat absorbed by the at least one sheet is transferred to the heat sink.

The heat radiation plate may be formed of graphene, graphite, copper or aluminum.

The ultrasonic probe may further include a protective layer provided at the front surface of the matching layer.

The protective layer may include an RF Shield or a Chemical Shield, and the protective layer may include a sheet formed of graphene or graphite.

In accordance with another aspect of the present disclosure, a method of manufacturing an ultrasonic probe includes preparing a backing layer, preparing a transducer layer at a front surface of the backing layer, and preparing a matching layer at a front surface of the transducer layer. The method may further include providing at least one sheet formed of grapheme on at least one of a front surface of the matching layer, in between the matching layer and the transducer layer, in between the transducer layer and the backing layer, a rear surface of the backing layer, and lateral sides of the matching layer, the transducer layer and the backing layer.

The method may further include forming a signal line connected to the at least one sheet. The signal line may transfer heat sensed from the sheet toward a backend of the ultrasonic probe so as to check a degree of heat emission of the ultrasonic probe.

At least one of the backing layer and the matching layer may be formed of graphene.

The method may further include forming a heat sink provided at the rear surface of the backing layer so as to dissipate heat generated from the ultrasonic probe to an outside.

The at least one sheet may extend to the heat sink so as to make thermal contact with the heat sink. The at least one sheet may transfer absorbed heat to the heat sink.

The method may further include providing at least one heat radiation plate that thermally connects the at least one sheet to the heat sink such that heat absorbed by the at least sheet is transferred to the heat sink.

The heat radiation plate may be formed of graphene, graphite, copper and aluminum.

The method may further include forming a protective layer provided at the front surface of the matching layer.

The protective layer may include an RF Shield or a Chemical Shield, and the protective layer may include a sheet formed of graphene or graphite.

In accordance with another aspect of the present disclosure, an ultrasonic probe system includes an ultrasonic probe, a signal output unit and a control unit. The ultrasonic probe may have a matching layer, a transducer layer provided at a rear surface of the matching layer, a backing layer provided at a rear surface of the transducer layer, at least one sheet formed of grapheme and provided on at least one of a front surface of the matching layer, in between the matching layer and the transducer layer, in between the transducer layer and the backing layer, a rear surface of the backing layer, and lateral sides of the matching layer, the transducer layer and the backing layer, and a signal line connected to the sheet to transmit information related to heat absorbed by the sheet. The signal output unit may output a signal configured to generate ultrasonic waves to the ultrasonic probe. The control unit may check a degree of heat emission of the ultrasonic probe based on the information transmitted through the signal line, and according to the degree of heat emission, control the signal output unit to adjust power of ultrasonic waves that are output from the ultrasonic probe.

As is apparent from the above, the state of heat emission of a probe can be monitored in real time.

In addition, a disadvantage associated with heat emission can be negated by dissipating heat generated from an ultrasonic probe by use of graphene or graphite.

In addition, the acoustic power of the ultrasonic probe can be increased by negating the heat emission related disadvantage.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a drawing illustrating a structure of an ultrasonic probe in accordance with an embodiment of the present disclosure.

FIG. 2 is a drawing illustrating a structure of a protective layer of the ultrasonic probe in accordance with an embodiment of the present disclosure.

FIGS. 3 to 5 are drawings illustrating a structure of an ultrasonic probe in accordance with another embodiment of the present disclosure.

FIG. 6 is a drawing illustrating a method of manufacturing an ultrasonic probe in accordance with an embodiment of the present disclosure.

### DETAILED DESCRIPTION

Reference will now be made in detail to the embodiments of the present disclosure, examples of which are illustrated in the accompanying drawings, wherein like reference numerals refer to like elements throughout.

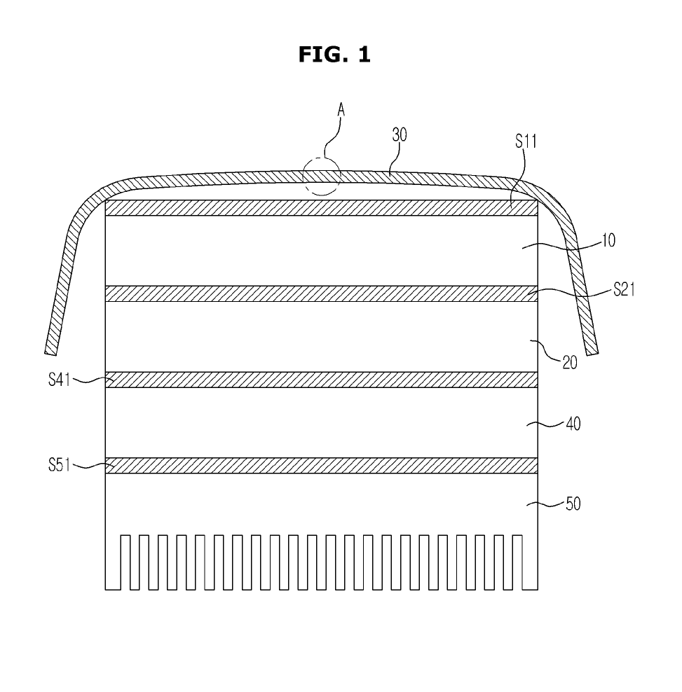

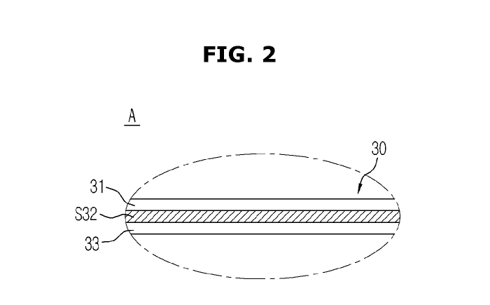

FIG. 1 is a drawing illustrating a structure of an ultrasonic probe in accordance with an embodiment of the present disclosure. FIG. 2 is a drawing illustrating a structure of a protective layer of the ultrasonic probe in accordance with an embodiment of the present disclosure.

Referring to FIG. 1, an ultrasonic probe in accordance with an embodiment of the present disclosure includes a transducer layer 20, a matching layer 10 provided at a front surface of the transducer layer 20, a protective layer 30 provided at a front surface of the matching layer 10, a backing layer 40 provided at a rear surface of the transducer layer 20, and a heat sink 50 provided at a rear surface of the backing layer 40. As an example of the transducer, a magnetrostrictive ultrasound transducer using magnetrostrictive effect of ferrite material, a capacitive micromachined ultrasonic transducer using vibration of several hundreds or several thousands of thin films that are microprocessed to transmit and receive ultrasonic waves, and a piezoelectric ultrasonic transducer using a piezoelectric effect of piezoelectric material may be used. Hereinafter, the piezoelectric ultrasonic transducer will be taken as an example in the following description.

Effect of voltage generation in a predetermined material in response to applied mechanical pressure and effect of mechanical deformation in response to applied voltage are respectively referred to as piezoelectric effect and inverse piezoelectric effect, and a material exhibiting such effects is referred to as a piezoelectric material.

That is, the piezoelectric material is a material which converts electrical energy into mechanical vibration energy or converts mechanical vibration energy into electrical energy.

The ultrasonic probe according to the present embodiment includes the transducer layer 20 formed of a piezoelectric material that generates ultrasonic waves in response to an electrical signal applied thereto by converting the electrical signal into mechanical vibration.

The piezoelectric material constituting the piezoelectric layer 20 may include lead zirconate titanate (PZT) ceramics, PZMT single crystals formed of a solid solution of lead magnesium niobate, and lead titanate or PZNT single crystals formed of a solid solution of lead zinc niobate and lead titanate.

In addition, the transducer layer 20 may have a single-layered or multi-layered stack structure. In general, impedance and voltage are easily controlled in the transducer layer 20 having a stack structure, so that excellent sensitivity, superior energy conversion efficiency, and soft spectrum may be obtained.

In addition, electrodes to which electrical signals are applied may be formed on the front and rear surfaces of the transducer layer 20. When the electrodes are formed on the front and rear surfaces of the transducer layer 20, one of the electrodes may be a ground electrode and the other may be a signal electrode. In addition, sheets S11, S21, S41 and S51, formed of graphene or graphite, which are described later, may serve as electrodes. Description of this will be described later in detail.

The matching layer 10 is provided on the front surface of the transducer layer 20. The matching layer 10 reduces an acoustic impedance difference between the transducer layer 20 and an object to match acoustic impedances of the transducer layer 20 and the object. Thus, ultrasonic waves generated in the transducer layer 20 are effectively transmitted to the object.

For this, the matching layer 10 may have a middle value between the acoustic impedances of the transducer layer 20 and the object.

The matching layer 10 may be formed of a glass or resin material.

Alternatively, the matching layer 10 may be formed of graphene. When the matching layer is formed of graphene, the matching layer may be used to connect electrical signals.

In addition, in order to change the acoustic impedance stepwise from the transducer layer 20 to the object, a plurality of matching layers 10 may be formed, and the matching layers 10 may be formed of different materials.

The transducer layer 20 and the matching layer 10 may be processed in a two-dimensional matrix array or in a one-dimensional array by a dicing process.

The protective layer 30 may be formed on the front surface of the matching layer 10. The protective layer 30 may be an RF Shield 31 capable of preventing leakage of a high-frequency component generated in the transducer layer 20 to the outside and blocking inflow of an external high-frequency signal.

Furthermore, the protective layer 30 may include a Chemical Shield 31 that is formed by coating or depositing a conductive material on a surface of a film having moisture resistance and chemical resistance so as to protect internal parts from water and chemicals used in disinfection, and the like.

The protective layer 30 is provided in the form having a sheet or film shaped graphene or graphite S32 coupled with the RF Shied or Chemical Shield 31 that is described above. That is, as shown in FIG. 2, a sheet or film shaped graphene or graphite S32 is formed on a film 33 serving as a base, and then the RF Shield or Chemical Shield 31 is formed on the graphene or graphite S32, thereby forming the protective layer 30. The protective layer including the sheet or film shaped graphene or graphite S32 may be used to connect electrical signals.

Although not shown in the drawings, a lens may be formed on the front surface of the protective layer 30. The lens may have a convex shape in an ultrasound-radiating direction so as to concentrate the ultrasonic waves, but the lens may have a concave shape if the speed of sound is lower than that in the human body.

The backing layer 40 is formed on the rear surface of the transducer layer 20. The backing layer 40 absorbs ultrasonic waves generated in the transducer layer 20 and proceeding in the backward direction of the transducer layer 20, thereby blocking reflection of ultrasound in the forward direction. Accordingly, image distortion may be prevented.

The backing layer 40 may be fabricated in a multi-layered structure in order to improve ultrasonic wave attenuation or blocking effects. The backing layer 40 may be formed of graphene or graphite. As the backing layer 40 is formed of graphene or graphite, the heat generated from the transducer layer 20 is effectively absorbed and transferred to the heat sink 50.

Electrodes, which apply an electrical signal to the transducer layer 20, may be formed on the front surface of the backing layer 40 that contacts the transducer layer 20. Sheets S formed of graphene or graphite, which will be described later, may serve as electrodes. Detailed description thereof will be made later.

The heat sink 50 provided at the rear surface of the backing layer 40 may include a plurality of plate-like fins formed of metal, such as aluminum, to disperse heat. Although not shown, in order to improve the heat radiation efficiency, a heat radiation fan may be provided adjacent to the heat radiation plate so as to dissipate heat dispersed from the fin of the heat sink 50 to the outside.

The ultrasonic probe includes sheets S that are provided in between the matching layer 10, the transducer layer 20 and the backing layer 40 that form the ultrasonic probe. The sheets S may be formed of graphene or graphite. In more detail, the sheet S formed of graphene or graphite may be provided on at least one of the front surface of the matching layer 10, in between the matching layer 10 and the transducer layer 20, in between the transducer layer 20 and the backing layer 40 and the rear surface of the backing layer 40. In addition, the sheet S may be provided at lateral sides of the backing layer, the transducer layer and the matching layer. On FIG. 1, the sheet S is illustrated as being provided on the front surface of the matching layer 10, in between the matching layer 10 and the transducer layer 20, in between the transducer layer 20 and the backing layer 40, and the rear surface of the backing layer 40, but the present disclosure is not limited thereto. For example, the sheet S may be provided on at least one position of the front surface of the matching layer 10, in between the matching layer 10 and the transducer layer 20, in between the transducer layer 20 and the backing layer 40, and the rear surface of the backing layer 40. As described above, the protective layer 30 may include the sheet S32 formed of graphene or graphite, and the backing layer 40 may be formed of graphene or graphite.

The graphite has a stacked structure, in each layer of which carbon atoms are arranged in a hexagonal honeycomb shape. The graphene is a layer separated from the graphite while having a slightest thickness. The graphene, which is an allotrope of carbon, represents nano material formed of carbon having an atomic number of 6, such as carbon nanotube and fullerene. The graphene has a two dimensional flat shape and a thickness of 0.2 nm while having superior physical and chemical stabilities. The graphene is known as having a thermal conductivity over twice as high as that of diamond, an electrical conductivity over 100 times as high as that of copper, and an electron mobility over 100 times as high as that of single crystal silicon that is mainly used in a semiconductor.

The ultrasonic probe in accordance with the embodiment of the present disclosure includes the sheets S formed of the graphene or graphite, to improve the heat radiation efficiency of the ultrasonic probe while providing interconnection of the ultrasonic probe or noise shielding effect. The sheets S formed of graphene or graphite may serve as electrodes. That is, the sheets S provided at the front surface and the rear surface of the transducer layer 20 may serve as a ground electrode or a signal electrode to apply an electrical signal to the transducer layer 20.

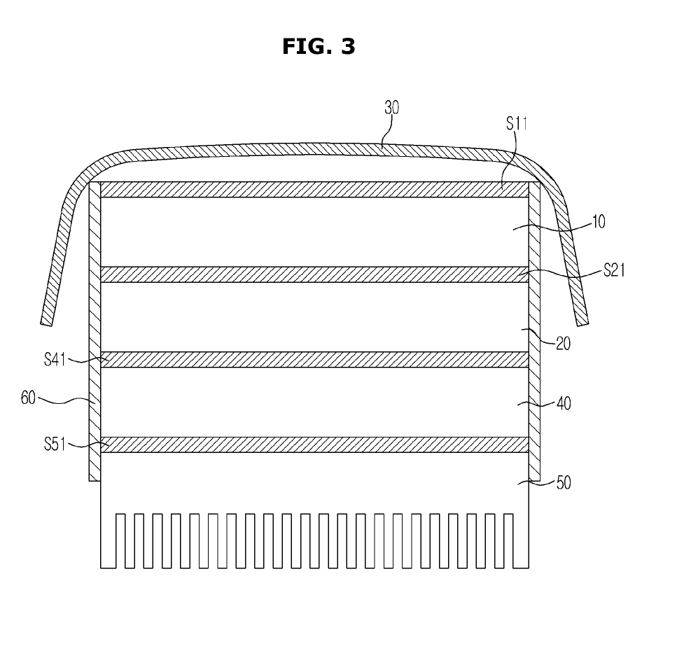

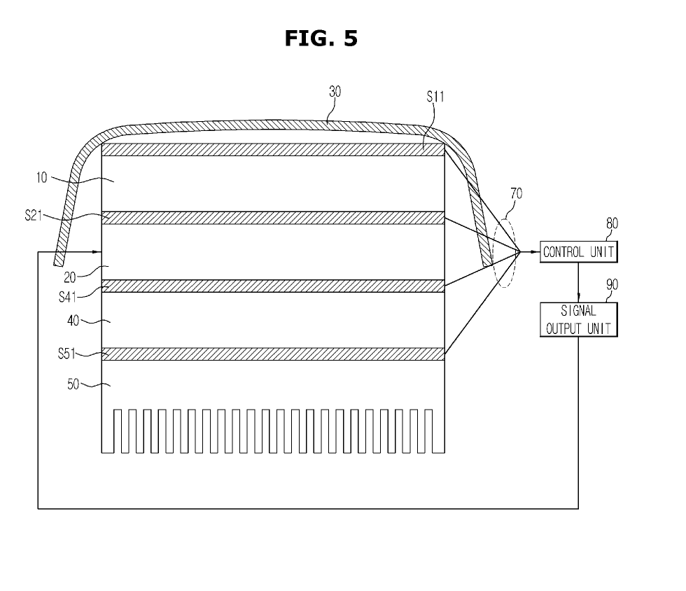

FIGS. 3 and 5 are drawings illustrating a structure of an ultrasonic probe in accordance with another embodiment of the present disclosure.

Referring to FIG. 3, a heat radiation plate 60 making contact with at least one sheet S may be provided at a lateral side of the ultrasonic probe while making contact with the heat sink 50.

The heat radiation plate 60 may allow heat absorbed from the sheets S formed of graphene or graphite to the heat sink 50 such that heat is dissipated to the outside through the heat sink 50. The heat radiation plate 60 may be formed of graphene, graphite, aluminum or copper. Alternatively, a heat pipe, instead of the heat radiation plate 60, may be used to transfer heat absorbed from the sheets S to the heat sink 50.

Referring to FIG. 3, the heat radiation plate 60 may be provided at opposite two lateral sides of the ultrasonic probe. Alternatively, the heat radiation plate 60 may be provided only at one lateral side of the ultrasonic probe different from FIG. 3.

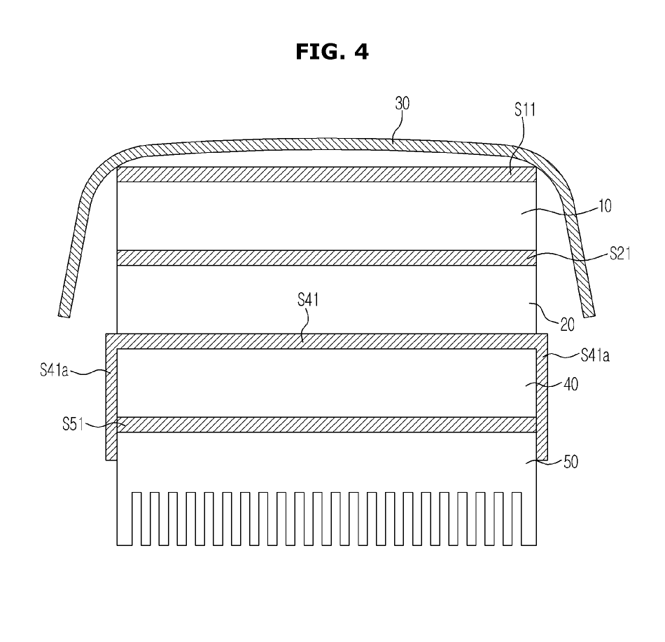

On FIG. 3, the heat radiation plate 60 is illustrated as separately provided to transfer the heat absorbed from the sheets S to the heat sink 50. On FIG. 4, portions S41a of the sheets S extend to the lateral sides of the ultrasonic probe to thermally make a direct contact with the heat sink 50, different from FIG. 3.

Although, on the drawing, only one of the sheets S, for example, the portions S41a, extends to the lateral sides of the ultrasonic probe to thermally make a directly contact with the heat sink 50, the present disclosure is not limited thereto. The remaining sheets S may extend in the same way as FIG. 4 to make contact with the heat sink 50.

As the sheets S formed of graphene or graphite having a superior thermal conductivity extend to make a direct contact with the heat sink 50, heat generated from the ultrasonic probe is effectively dissipated to the outside.

FIG. 5 illustrates a signal line 70 connected to the sheets S.

Information related to heat that is generated from the ultrasonic probe and absorbed into the sheets S is transmitted to a control unit 80 of an ultrasonic probe system through the signal line 70 connected to the sheets S.

The control unit 80 checks a degree of heat emission of the ultrasonic probe based on the information transmitted through the signal line 70 in real time, and according to the degree of heat emission, adjusts the operation of the ultrasonic probe.

For example, the control unit 80 checks a degree of heat emission of the ultrasonic probe based on the information transmitted through the signal line 70, and if the degree of heat emission of the ultrasonic probe exceeds a predetermined threshold value, controls a signal output unit 90, which outputs a signal for generating ultrasonic waves to the ultrasonic probe, such that the heat emission of the ultrasonic probe is reduced. In addition, if the heat emission of the ultrasonic probe is below the predetermined threshold value, the control unit 80 may control the signal output unit 90 such that intensities of ultrasonic waves output from the ultrasonic probe are increased even if the heat emission of the ultrasonic probe is increased to some degrees. That is, the acoustic power of the ultrasonic probe and the heat emission state of the ultrasonic probe are in a trade off relation between each other.

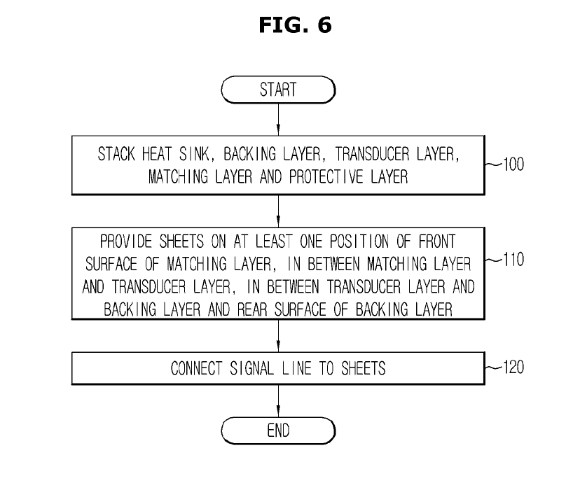

FIG. 6 is a drawing illustrating a method of manufacturing an ultrasonic probe in accordance with an embodiment of the present disclosure.

The heat sink 50, the backing layer 40, the transducer layer 20, the matching layer 10 and the protective layer 30 that form the ultrasonic probe are stacked up against one another (100).

The heat sink 50 is provided at the rear surface of the backing layer 40, the transducer layer 20 is provided on the front surface of the backing layer 40, the matching layer 10 is provided on the transducer layer 20, and the protective layer 30 is provided on the front surface of the matching layer 10.

As describe above, the backing layer 40 may be formed of graphene or graphite. The protective layer 30 may be provided in the form having the sheet or film shaped graphene or graphite S32 coupled to the RF Shield or Chemical Shield 31 described above. That is, as shown in FIG. 2, a sheet or film shaped graphene or graphite S32 is formed on the film 33 serving as a base, and then the RF Shield or Chemical Shield 31 is formed on the graphene or graphite S32, thereby forming the protective layer 30. The sheets S formed of graphene or graphite may be provided on at least one position of the front surface of the matching layer 10, in between the matching layer 10 and the transducer layer 20, in between the transducer layer 20 and the backing layer 40 and the rear surface of the backing layer 40 (110). The sheets S formed of graphene or graphite may extend to the lateral sides of the ultrasonic probe to make a contact with the heat sink 50. In addition, the heat radiation plate 60 making contact with at least one of the sheets S while making contact with the heat sink 50 may be provided at a lateral side of the ultrasonic probe. The heat radiation plate 60 may be formed of graphene, graphite, aluminum or copper. Alternatively, a heat pipe, instead of the heat radiation plate 60, may be used to transfer heat absorbed from the sheets S to the heat sink 50.

The signal line 70 is connected to the sheets S after the sheets S are provided (120). Information related to heat that is generated from the ultrasonic probe and absorbed into the sheets S is transmitted to the control unit 80 of an ultrasonic probe system through the signal line 70 connected to the sheets S.

The control unit 80 checks a degree of heat emission of the ultrasonic probe based on the information transmitted through the signal line 70 in real time, and according to the degree of heat emission, adjusts the operation of the ultrasonic probe.

For example, the control unit 80 checks a degree of heat emission of the ultrasonic probe based on the information transmitted through the signal line 70, and if the degree of heat emission of the ultrasonic probe exceeds a predetermined threshold value, controls the signal output unit 90, which outputs a signal for generating ultrasonic waves to the ultrasonic probe, such that the heat emission of the ultrasonic probe is reduced. In addition, if the heat emission of the ultrasonic probe is below the predetermined threshold value, the control unit 80 may control the signal output unit 90 such that intensities of ultrasonic waves output from the ultrasonic probe are increased even if the heat emission of the ultrasonic probe is increased to some degrees. That is, the acoustic power of the ultrasonic probe and the heat emission state of the ultrasonic probe are in a trade off relation between each other.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. An ultrasonic probe comprising:
a matching layer;
a transducer layer provided at a rear surface of the matching layer; and
a backing layer provided at a rear surface of the transducer layer,
wherein the ultrasonic probe further comprises at least one sheet that is formed of graphene and provided on at least one of a front surface of the matching layer, in between the matching layer and the transducer layer, in between the transducer layer and the backing layer, a rear surface of the backing layer, and lateral sides of the matching layer, the transducer layer and the backing layer.

2. The ultrasonic probe of claim 1, further comprising a signal line connected to the at least one sheet,
wherein the signal line transmits heat sensed from the sheet toward a backend of the ultrasonic probe so as to check a degree of heat emission of the ultrasonic probe.

3. The ultrasonic probe of claim 1, wherein at least one of the backing layer and the matching layer is formed of graphene.

4. The ultrasonic probe of claim 1, further comprising a heat sink provided at the rear surface of the backing layer so as to dissipate heat generated from the ultrasonic probe to the outside.

5. The ultrasonic probe of claim 4, wherein the at least one sheet extends to the heat sink so as to make thermal contact with the heat sink, and transfers absorbed heat to the heat sink.

6. The ultrasonic probe of claim 4, further comprising at least one heat radiation plate that thermally connects the at least one sheet to the heat sink such that heat absorbed by the at least one sheet is transferred to the heat sink.

7. The ultrasonic probe of claim 6, wherein the heat radiation plate is formed of graphene, graphite, copper or aluminum.

8. The ultrasonic probe of claim 1, further comprising a protective layer provided at the front surface of the matching layer.

9. The ultrasonic probe of claim 8, wherein:
the protective layer includes an RF Shield or a Chemical Shield; and
the protective layer includes a sheet formed of graphene or graphite.

10. A method of manufacturing an ultrasonic probe, the method comprising:
preparing a backing layer;
preparing a transducer layer at a front surface of the backing layer; and
preparing a matching layer at a front surface of the transducer layer,
wherein the method further comprises providing at least one sheet formed of grapheme on at least one of a front surface of the matching layer, in between the matching layer and the transducer layer, in between the transducer layer and the backing layer, a rear surface of the backing layer, and lateral sides of the matching layer, the transducer layer and the backing layer.

11. The method of claim 10, further comprising forming a signal line connected to the at least one sheet,
wherein the signal line transfers heat sensed from the sheet toward a backend of the ultrasonic probe so as to check a degree of heat emission of the ultrasonic probe.

12. The method of claim 10, wherein at least one of the backing layer and the matching layer is formed of graphene.

13. The method of claim 10, further comprising forming a heat sink provided at the rear surface of the backing layer so as to dissipate heat generated from the ultrasonic probe to an outside.

14. The method of claim 13, wherein:
the at least one sheet extends to the heat sink so as to make thermal contact with the heat sink; and
the at least one sheet transfers absorbed heat to the heat sink.

15. The method of claim 13, further comprising providing at least one heat radiation plate that thermally connects the at least one sheet to the heat sink such that heat absorbed by the at least sheet is transferred to the heat sink.

16. The method of claim 15, wherein the heat radiation plate is formed of graphene, graphite, copper and aluminum.

17. The method of claim 10, further comprising forming a protective layer provided at the front surface of the matching layer.

18. The method of claim 8, wherein:
the protective layer includes an RF Shield or a Chemical Shield; and
the protective layer includes a sheet formed of graphene or graphite.

19. An ultrasonic probe system comprising:
an ultrasonic probe having a matching layer, a transducer layer provided at a rear surface of the matching layer, a backing layer provided at a rear surface of the transducer layer, at least one sheet formed of grapheme and provided on at least one of a front surface of the matching layer, in between the matching layer and the transducer layer, in between the transducer layer and the backing layer, a rear surface of the backing layer, and lateral sides of the matching layer, the transducer layer and the backing layer, and a signal line connected to the sheet to transmit information related to heat absorbed by the sheet;
a signal output unit outputting a signal configured to generate ultrasonic waves to the ultrasonic probe; and
a control unit checking a degree of heat emission of the ultrasonic probe based on the information transmitted through the signal line, and according to the degree of heat emission, controlling the signal output unit to adjust power of ultrasonic waves that are output from the ultrasonic probe.
